(19) United States
(12) Patent Application Publication
(10) Pub. No.: US 2026/0234528 A1
(43) Pub. Date: Aug. 13, 2026

(54) Device For Cultivating Cell Biological Samples In A Culture Insert

(71) Applicant: ibidi GmbH, Gräfelfing (DE)

(72) Inventors: Nina HUBER, Dachau (DE); Georg LUBINS, München (DE); Trung PHAM, München (DE); Elias HORN, Feucht (DE); Jan SCHWARZ, Bingen (DE); Roman ZANTL, Gräfelfing (DE); Juliane HÖNIG, Bad Säckingen (DE)

(21) Appl. No.: 19/537,053

(22) Filed: Feb. 11, 2026

(30) Foreign Application Priority Data
Feb. 12, 2025 (EP) 25157334.1

(51) Int. Cl. C12M 1/12; G06Q 10/06; G06Q 10/10

(52) U.S. Cl. CPC C12M 25/04; G06Q 10/06; G06Q 10/10

(57) ABSTRACT

A device for cultivating cell biological samples in a culture insert includes a base element in which a well with a bottom is formed. The well is configured for arranging the culture insert in the well. The device further includes a positioning element configured to provide a first receiving position and a second receiving position for the culture insert. The culture insert is arranged in one of the first receiving position or in the second receiving position on the positioning element so that the culture insert is arranged in the well. In the first receiving position, a distance between the bottom of the well and a bottom of the culture insert is smaller than in the second receiving position.

No new matter is added via these amendments.

### RELATED APPLICATIONS

This application claims the benefit of European Patent Application No. 25157334.1, filed Feb. 12, 2025, the entire contents of which is hereby incorporated by reference.

### TECHNICAL FIELD

The present disclosure relates to a device for cultivating cell biological samples.

### BACKGROUND

Transwell systems or membrane insert systems are used for cellular interface experiments such as co-cultures and transmigration experiments. Almost all membrane inserts, also known as culture inserts, are based on the same principle: a porous membrane serves as the bottom of the membrane insert, forming a pot with a porous bottom that is permeable to cells and molecules. This pot can be inserted into a reservoir containing nutrient fluid. The porous membrane then serves as a scaffold or substrate for cell adhesion, allowing cells to form single- or multi-layered structures on the membrane, while at the same time allowing nutrients from the nutrient fluid to diffuse through to supply the cells with nutrients. The reservoir in which the membrane insert is placed must provide sufficient space for a nutrient solution under and around the membrane insert, as the cultivation of living cells below the bottom requires sufficient nutrient exchange. Therefore, the distance between the bottom of the reservoir and the porous membrane must not fall below a certain threshold value during cultivation.

At the same time, this threshold value for the distance to the bottom of the reservoir significantly limits the ability to examine the cells on the porous membrane using standard objectives when the cells are to be examined through the bottom using inverse microscopy. One way to circumvent this problem is to remove the porous membrane from the membrane insert and position it on a microscope cover glass. While this is possible for the endpoint analysis of individual samples, process control and automation of microscopy cannot be achieved in this way.

Therefore, there is a need for a simple and user-friendly device that provides a suitable arrangement for changing the position of the membrane insert between an ideal position for cultivating, i.e., sufficient supply of nutrient solution below the porous membrane due to a minimum distance between the reservoir bottom and the porous membrane, and an ideal position for imaging, i.e., with the smallest possible distance between the objective and the cells or samples. With the solutions currently available, such a device cannot be satisfactorily achieved.

### SUMMARY

Provided herein is a device for cultivating cell biological samples, which includes a simple and user-friendly mechanism for switching between a position of the membrane insert for cultivating and a position for microscopy.

### BRIEF DESCRIPTION OF THE DRAWINGS

Embodiments of the present disclosure are explained in more detail with reference to the following exemplary Figures.

FIG. 1 shows a first device for cultivating cell biological samples;

FIG. 2 shows a second device for cultivating cell biological samples;

FIGS. 3A and 3B show a third device for cultivating cell biological samples together with a detailed view;

FIGS. 4A and 4B show a fourth device for cultivating cell biological samples together with a detailed view;

FIGS. 5A and 5B show a fifth device for cultivating cell biological samples together with a detailed view;

FIG. 6 shows a positioning element of a sixth device for cultivating cell biological samples;

FIG. 7 shows a positioning element for a seventh device for cultivating cell biological samples;

FIG. 8 shows a detailed view of the stand according to the seventh device for cultivating cell biological samples; and

FIGS. 9A and 9B show a detailed view of the seventh device for cultivating cell biological samples with the positioning element in the first position and the second position.

In the following and in the figures, unless otherwise specified, the same reference characters are used for the same or corresponding elements in the various embodiments.

### DETAILED DESCRIPTION

According to some embodiments, a device for cultivating cell biological samples is provided, the device comprising: a base element in which a well with a bottom is formed, wherein the culture insert can be arranged in the well, and a positioning element that provides a first receiving position and a second receiving position for the culture insert, wherein the culture insert can be arranged in the first receiving position and in the second receiving position on the positioning element so that the culture insert is arranged in the well, and wherein, in the first receiving position, a distance between the bottom of the well and a bottom of the culture insert is smaller than in the second receiving position.

The device thus provides two different receiving positions for the culture insert, which is arranged in the well in both positions. This means that the culture insert protrudes into the well in both receiving positions. The first receiving position is characterized by a smaller distance between the bottom of the culture insert and the bottom of the well than the second receiving position. Consequently, the first receiving position is particularly suitable for microscopy and the second receiving position is intended for cultivation. These two receiving positions are provided by the positioning element. It is therefore possible to switch between the two receiving positions with the aid of the positioning element, and it is not necessary to lift or remove the culture insert from the well. This simplifies switching between the two receiving positions and makes it more user-friendly.

The first receiving position can be a position in which the distance between the bottom of the culture insert and the bottom of the well is zero. This means that the bottom of the culture insert rests on the bottom of the well. Alternatively, the distance between the bottom of the culture insert and the bottom of the well may be less than 1 mm. In the second receiving position, the distance between the bottom of the well and the bottom of the culture insert is greater than in the first receiving position. This allows a nutrient solution to flow freely between this gap and ensures nutrient exchange through the porous membrane. In the second receiving position, the gap can be 1 mm or more, in particular 2 mm or more, or even 3 mm or more.

The culture insert can also be referred to as membrane insert.

The bottom of the well can be transparent. This allows inverse microscopy through the bottom. Samples located in the culture insert can thus be easily examined. The bottom of the well can be transparent, particularly in the visible wavelength range. Instead or in addition, transparency in the near-infrared wavelength range (e.g., between 800 nm and 1500 nm) may also be provided. Together with the distance between the bottom of the culture insert and the bottom of the well of less than 1 mm in the first receiving position, high resolution can be achieved in inverse microscopy through the bottom.

The bottom of the well can have a thickness between 100 μm and 2.0 mm, in particular between 160 μm and 190 μm, more particularly between 165 μm and 175 μm, for example 170 μm. This minimizes the distance between the bottom of the culture insert and an objective in the first receiving position, which allows high resolution. The bottom of the well may comprise or be made of glass or a plastic such as a polymer film. The bottom of the well may have the optical properties, for example with regard to birefringence and autofluorescence, of a cover glass (such as D 263 Schott glass, No. 1.5H). The well may have the shape of a truncated cone or a cylinder. A cross-section of the well may be circular. The base element may be a multi-well plate in which several wells are arranged in a regular structure. An example is a plate with 24 wells arranged in six columns and four rows. A plate with six wells arranged in three columns and two rows is also possible. Compared to a single well, a multi-well plate offers the advantage that a plurality of samples can be cultivated simultaneously, thereby increasing throughput. All wells of the multi-well plate may be equally formed.

The average diameter of the well is, for example, 16.0 mm. The depth of the well can be approximately 18.0 mm.

The culture insert may have a cylindrical basic shape. The average outer diameter of the culture insert may be 10.0 mm. There may be a distance of approximately 3 mm between the culture insert and an inner wall of the well when the culture insert is centered within the well.

Furthermore, the culture insert may comprise fastening element(s) for arranging or fastening it to the positioning element.

The bottom of the culture insert may comprise or be a porous membrane. The membrane may be permeable to cells or molecules, as well as to liquids. The membrane may serve as a scaffold or substrate for cell adhesion, allowing cells to form single-layer and multi-layer structures on the membrane.

The bottom of the culture insert may have a thickness between 1 μm and 500 μm. In particular, the thickness may be between 10 μm and 100 μm. Example values are 12 μm and 50 μm.

If the objective is brought as close as possible to the bottom of the well, the minimum distance between the objective and the cells on the bottom of the culture insert corresponds to the sum of the thickness of the bottom of the well and the thickness of the bottom of the culture insert.

The culture insert can be hooked into the positioning element, in particular in the first and/or second receiving position. The culture insert can comprise a protrusion. The protrusion can interact with a corresponding counterpart on the positioning element. In this way, the culture insert can be hooked into the positioning element.

The positioning element may have two different levels that define the first receiving position and the second receiving position, and may be formed such that a change between the first receiving position and the second receiving position is effected by a relative movement between the positioning element and the culture insert.

The change between the two receiving positions is effected by a simple relative movement. In particular, the positioning element may be stationary relative to the base element. The culture insert may be formed such that the change between the two receiving positions is effected by a movement of the culture insert, wherein the movement comprises a displacement (translational) and/or a lifting or lowering and/or a rotation.

For example, the two levels can be formed on an upper edge of the well and connected by a step. Here, a change from the first receiving position to the second receiving position would be effected by rotating the culture insert in combination with lifting it over the step between the two levels.

A slope connecting the two levels can be provided between the two levels. The angle of inclination of the slope may be greater than 0° and less than 90°. In particular, the angle of inclination may be between 20° and 70°, between 30° and 60°, or between 40° and 50°. Thanks to the slope, the culture insert does not need to be lifted in order to be moved into the second receiving position. During rotation, the difference in height between the two receiving positions is overcome by pushing up over the slope.

The positioning element is movable relative to the base plate and can be brought into a first position and a second position, wherein the first position defines the first receiving position and the second position defines the second receiving position. This means that the positioning element can be brought into a first position and a second position relative to the base element. The first position of the positioning element defines the first receiving position for the culture insert. The second position of the positioning element defines the second receiving position for the culture insert. Therefore, the first position and the second position are different. In particular, the base element may have two levels that define the first position and second position for the positioning element.

This represents an alternative way of implementing a simple change from the first to the second receiving position. Whereas in the previous example the positioning element is stationary relative to the base element and the change is effected by moving the culture insert, in this example the positioning element is moved relative to the base element.

In particular, the culture insert can be stationary relative to the positioning element. This has the particular advantage that the culture insert does not have to be moved separately. This reduces the risk of accidental contamination of the samples or cells in the culture insert, which could otherwise occur due to contact between the culture insert and a user moving the culture insert.

The movement of the positioning element between the two receiving positions can be a purely vertical movement (parallel to a longitudinal axis of the well). Alternatively, the movement of the positioning element can be a combination of a horizontal and a vertical movement. A particular advantage of purely vertical movement is that there are no strict restrictions on the height difference between the first receiving position and the second receiving position. With horizontal movement, the range is instead limited by the original distance between the side wall of the well and the culture insert.

According to some embodiments, the positioning element provides the two receiving positions. The positioning element itself may have means for arranging the culture insert in two different positions on the positioning element in order to realize the two receiving positions. Alternatively, the positioning element may be movable into two different positions relative to the base element, thus providing the two receiving positions. The advantage of increased user-friendliness is achieved equally by these two configurations.

The device may further comprise a ramp, wherein the positioning element can be brought from the first position to the second position via the ramp. In this context, a ramp refers to a surface that is inclined at an angle greater than 0° and less than 90° relative to a horizontal plane. This allows a height difference to be overcome by pushing over the ramp. Here, this is the height difference between the first position and the second position of the positioning element. The angle of the ramp may be between 20° and 70°, between 30° and 60°, or between 40° and 50°.

Thanks to the ramp, the positioning element can be moved from the first position to the second position solely by applying a lateral force. The vertical displacement of the positioning element is achieved by pushing it up the ramp. This greatly simplifies the change between the two positions and thus between the two receiving positions for the culture insert. Furthermore, it is easier to automate the change. A robot designed for this purpose only needs to be trained to move the positioning element sideways in order to perform the change. This is easier to implement than lifting or a combination of lifting and sideways movement.

The ramp can be part of the base element. The positioning element can be in contact with a part of the base element. This part can then be pushed over the ramp to move the positioning element from the first to the second position. It is conceivable, for example, that the positioning element comprises a stand with which the positioning element stands on the base element, and the stand can be pushed over the ramp.

The ramp can be arranged on a horizontal section of the base element. The edge of the well can be chamfered. This bevels the edge and can itself form a ramp over which the stand can slide.

The positioning element can also be brought from the second position to the first position via the ramp.

Alternatively, the ramp can be an additional movable element of the device that can be displaced relative to the base element, wherein the positioning element can be brought from the first position to the second position by moving the ramp. The ramp can only be moved horizontally.

Given that the ramp is an additional element, it is also referred to as a ramp element. To switch between the first receiving position and the second receiving position, only a displacement (horizontal, no vertical movement) of the ramp element is necessary. The displacement provides a switch between the first position and the second position of the positioning element. In particular, it is not necessary to actively move the positioning element, which further simplifies the switch between the two receiving positions.

The movement of the positioning element caused by the displacement of the ramp can only be vertical, i.e., along a longitudinal axis of the well. In other words, a direction of movement of the ramp and a direction of movement of the positioning element can be perpendicular to each other.

The device may comprise a plurality of ramp elements, each ramp element being displaceable relative to the base element. Each ramp element may be displaceable separately, i.e., independently of the other ramp elements.

In a device with a ramp, the positioning element may be arranged above the base element, with the ramp being arranged between the base element and the positioning element, and with the positioning element being in contact with the ramp. In particular, the positioning element may rest partially on the ramp. The ramp may be arranged directly on the base element.

This arrangement, in which the base element, ramp or ramp element, and positioning element are arranged one above the other in this order, offers the advantage that it is possible to switch between the two positions without requiring large force. Strictly speaking, the force required is essentially determined by the mass of the positioning element.

The positioning element may be arranged above the base element and may have a stand with at least a partially beveled underside, wherein the beveled part of the underside of the stand is in contact with an edge of the well and partially protrudes into the well when the positioning element is in the first position. Alternatively, the base element may comprise a recess adjacent to the well, wherein the beveled underside of the stand protrudes into the recess when the positioning element is in the first position. An advantage of using the well is that the positioning element can be used with a multi-well plate. An additional recess is then not necessary.

Here, the ramp is implemented by the beveled underside of the stand. Moving the positioning element causes the beveled underside to slide over the edge of the well, changing the distance between the base element and the positioning element located above it. In this way, the positioning element is brought from the first position to the second position and vice versa. Neither a separate ramp element nor a ramp as part of the base element is necessary in this case. This design therefore represents a simple and easy-to-manufacture option for enabling the change between the two receiving positions for culture insert. In this implementation, the movement of the positioning element consists of a horizontal displacement and a vertical lifting or lowering.

A first section of the underside may be beveled, and a second section adjoining the first section may be horizontal. In the second position, the second section may rest on the edge of the well. In the first position, as described above, the first section may rest on the edge of the well.

The culture insert can be locked in the second receiving position by requiring increased force to move the culture insert into the first receiving position. The positioning element can be locked in the second position by requiring increased force to move the positioning element into the first position. For example, there can be a positive connection between the positioning element and the base element in the second position. A positive connection between the culture insert and the positioning element is also possible when the culture insert is in the second receiving position.

This locking prevents the culture insert from being unintentionally moved from the second receiving position to the first receiving position (e.g., by a slight impact against the device) or the positioning element from being unintentionally moved from the first position to the second position. For example, without locking, the positioning element could unintentionally slide down the ramp.

The increased force required is to be understood as meaning that a force must be applied that exceeds the static friction between the ramp and the positioning element. For example, an additional elevation may be provided on the ramp, which must be overcome in order to move the positioning element from the second position to the first position. Alternatively, the ramp may include a section that has higher friction (especially higher sliding friction) with the positioning element. The section of the ramp may have a surface made of rubber or another material that increases the friction.

The underside of the stand of the positioning element may have three sections:
a first section that is inclined at a first angle relative to a horizontal plane,
a second section that adjoins the first section and is inclined at a second angle, which is opposite in sign to the first angle, to the horizontal plane, and
a third section that adjoins the second section and is formed parallel to the horizontal plane,
wherein, in the first receiving position, the first section is in contact with the edge of the well, and wherein, in the second receiving position, the third section is in contact with the edge of the well. The horizontal is, in particular, perpendicular to the vertical or to the longitudinal axis of the well.

These three sections give the underside of the stand a triangular protrusion that can be described as a tooth. In the second position of the positioning element, the third section rests on the edge of the well. To move the positioning element into the first position, the tooth must be overcome, which requires increased force. This locks or engages the positioning element in the second position and prevents it from accidentally falling into the first position. Once the tooth has been overcome, the positioning element slides along the first section into the first position.

The third section can also be inclined at an angle of no more than 10° to the horizontal. This also ensures, in the second position of the positioning element, that the third section rests on the edge of the well and cannot slip. If the angle is sufficiently small, the static friction between the stand and the base element is sufficient to prevent slipping.

This design is particularly suitable if the base element is a multi-well plate with adjacent wells. In the second position of the positioning element, the third section rests on the surface between the adjacent wells, which separates the two wells, and the tooth protrudes into the adjacent well. The surface between adjacent wells is also referred to as a partition wall.

The first angle can be between 50° and 60°, in particular 54°. The second angle can be between 65° and 75°, in particular 70°.

The specified values for the first and second angles may be optimal in terms of practicality. In particular, these angles provide secure locking of the positioning element in the second position, and an appropriate amount of force is required to move the positioning element from the first to the second position.

Viewed from the third section, the height of the tooth can be between 0.7 and 0.9 mm, in particular 0.8 mm. The tooth can be rounded at its tip. The rounding ensures a more even movement of the positioning element when the displacement occurs over the tooth. In addition, the height of the tooth is chosen so that the height difference to be overcome is not too great. The third section can have a length of approximately 1 mm as seen from the second section.

With the ramp, the positioning element only needs to be moved sideways to switch between the two positions. The first section serves as a ramp, which additionally converts the lateral movement into a vertical movement. This movement can be automated relatively easily with a robot, as no complex movement sequences are required.

The device may also have one lateral limiting element or more lateral limiting elements to limit the horizontal movement of the positioning element between the first position and the second position. The at least one lateral limiting element ensures that the positioning element cannot be moved beyond the first position and the second position. At the same time, the first and second positions are clearly defined by the stop at the at least one lateral limiting element, allowing a user to clearly identify the correct position of the positioning element. The at least one lateral limiting element is also helpful in the context of the automation with a robot already discussed, because it specifies the two positions of the positioning element and they do not have to be precisely implemented in the programming of the robot.

The at least one lateral limiting element may be attached to the base element. The lateral limiting element may form a frame extending upward from the base element, whereby the positioning element can be moved within the frame.

The positioning element may have a through hole formed which is formed for arranging the culture insert, wherein the through hole comprises an additional bulge. The bulge allows the shape of the through hole to deviate from a circular or elliptical shape.

The bulge can be used to introduce culture medium past the culture insert into the well using a pipette, especially when the culture insert is in the second receiving position. The bulge provides additional space for the pipette and simplifies the addition of culture medium.

The positioning element may have two stands with the three sections. The positioning element may have four stands, at least two of which have the three sections. The remaining stands may have a flat underside and/or a beveled underside. The arrangement of the stands can be symmetrical with respect to a central axis of the positioning element.

The provision of four stands increases the stability of the device because the positioning element rests stably on the base element. The provision of two of the stands with the three sections ensures that the positioning device is not blocked on one side and unintentionally rotated relative to the base element. Since the stands with the three sections are more delicate to manufacture than stands with a flat underside or a beveled underside, the manufacture of the positioning element is simplified if not all stands comprise the three sections.

The base element may comprise or consist of a plastic. In particular, it may comprise plastics such as COC (cyclo-olefin copolymer), COP (cyclo-olefin polymer), PC (polycarbonate), PS (polystyrene), PE (polyethylene), PMMA (polyethylene methacrylate) or a transparent thermoplastic or elastomer. The positioning element may comprise or consist of a plastic material. In particular, the positioning element may comprise or consist of PS, polypropylene (PP) or polyethylene terephthalate (PET). The choice of these plastics offers the advantage that both elements can be sterilized and are therefore reusable.

The base element and/or the positioning element may be an injection-molded part. By using the aforementioned materials, the base element or the positioning element can be produced cost-effectively and in large quantities with consistent quality. Alternatively, the positioning element and/or the base element may be manufactured using 3D printing. 3D printing has the advantage that delicate and complex structures can be reliably produced. The base element and/or the positioning element can be made in one piece.

Alternatively, the base element can comprise or consist of a glass.

The glass or plastic for the base element can exhibit, in particular, the birefringence and autofluorescence of a Schott cover glass (such as D 263 Schott glass, No. 1.5H).

The device may comprise a lid that can be placed on the base element. The lid may be form-fitting. In particular, when the lid is in place, the positioning element is located between the base element and the lid. The lid shields the culture insert and the cells contained therein from external environmental influences, thus protecting them from contamination.

The present disclosure further provides a system comprising the above-described device for cultivating cell biological samples and a culture insert.

The culture insert may comprise: a bottom with a porous membrane that is permeable to cells or molecules; and a fastening element for arranging the culture insert on the positioning element. The culture insert may be a disposable item for single use.

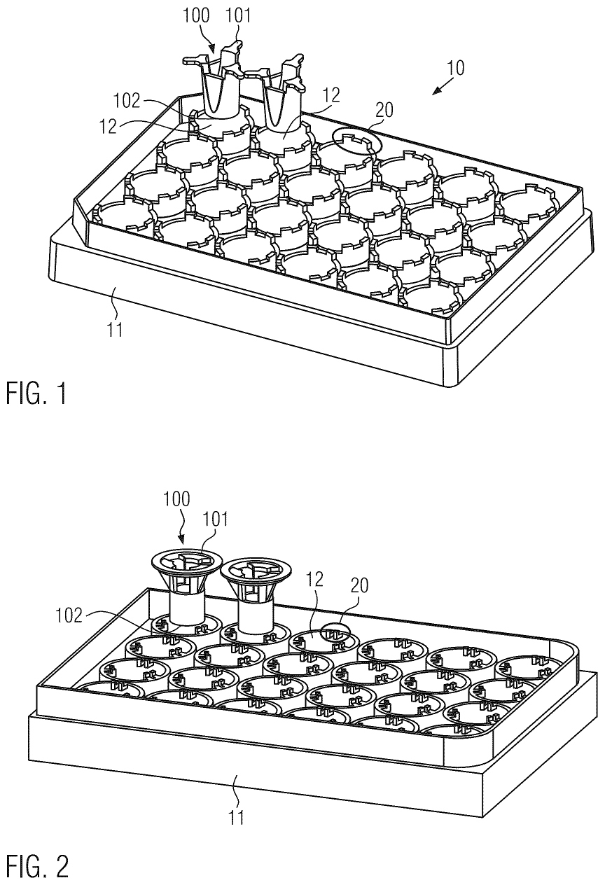

Referring not to the figures, FIG. 1 shows a first device 10 for cultivating cell biological samples in a culture insert 100. This device comprises a base element 11 in which a well 12 with a bottom is formed. In particular, the base element can be a multi-well plate, which in this example has 24 wells arranged in a square grid of six columns and four rows. All wells of the multi-well plate may be formed identically. It is understood that the base element 12 is not limited to the shape and number of wells shown.

In the following, a well 12 and a culture insert 100 arranged therein are discussed, and the explanations given therefore apply in the same way to the other wells of the multi-well plate. In addition, the other devices described in accordance with the following figures are also based on such a multi-well plate as the base element 11, without this being necessarily mentioned.

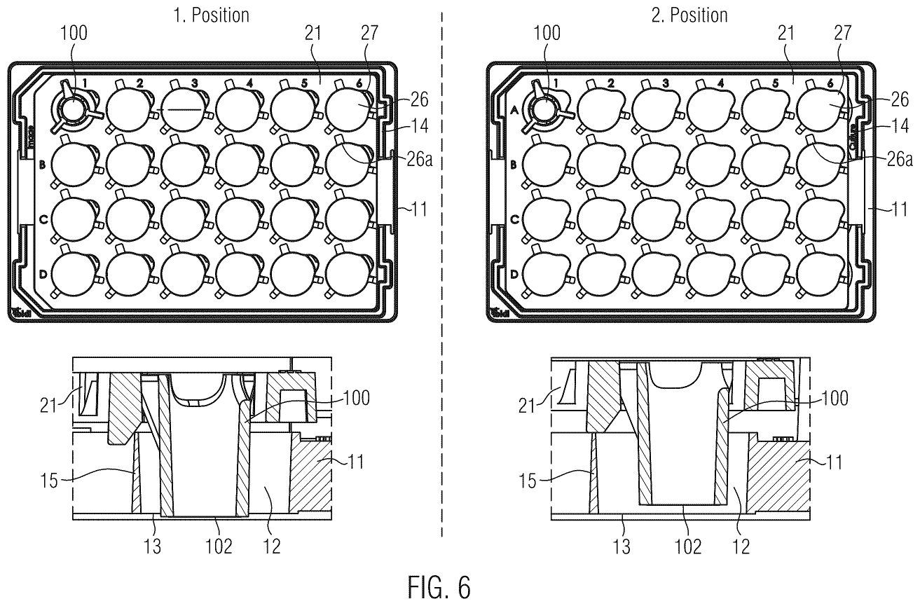

Furthermore, the device 10 comprises a positioning element 20 that provides a first receiving position and a second receiving position for the culture insert 100. In the first receiving position, the distance between the bottom of the well 12 and a bottom 102 of the culture insert 100 is smaller than in the second receiving position. A pictorial representation of the distance can be seen in FIG. 6, which shows the culture insert in the first and second receiving positions. To achieve this, the positioning element 20 has two levels. More specifically, the positioning element 20 is formed around the well as a structure comprising two levels. The higher level defines the second receiving position, while the lower level defines the first receiving position. The positioning element 20 is stationary relative to the base element 11.

The culture insert 100 can be arranged in the well 12 so that it protrudes into the well 12. For this purpose, the culture insert 100 comprises three fastening elements 101 with which it can be arranged on the positioning element at three support points. The arrangement can in particular be a fastening or a hooking. In the example shown, the fastening element is a projection or hook with which the culture insert 100 can be hooked onto the positioning element 20. In the second receiving position, the culture insert 100 is hooked onto the higher level. In the first receiving position, the culture insert 100 is hooked onto the lower level.

The first receiving position can be a position in which the distance between the bottom of the culture insert and the bottom of the well is as small as possible. In particular, the distance is zero in the first receiving position, i.e., the bottom of the culture insert rests on the bottom of the well. A distance of less than 1 mm is also possible. In the second receiving position, the distance between the bottom of the recess and the bottom of the culture insert is greater. This allows a nutrient solution to flow well between this distance and ensures a nutrient exchange through the porous membrane. In this distance, the distance can be 1 mm or more, in particular also 2 mm or more, or even 3 mm or more. Therefore, the first receiving position can also be referred to as the microscopy position. The second receiving position can be referred to as the cultivating position.

In order to get from the first receiving position to the second receiving position, the culture insert 100 must be raised vertically relative to the positioning element 20 and rotated relative to a longitudinal axis of the well 12. The positioning element 20 is stationary relative to the base element 11. However, it is not sufficient to simply rotate the culture insert 100 to get from the second receiving position to the first receiving position. This is because an additional elevation is provided between the two levels in which the culture insert 100 is hooked in. This blocks the culture insert 100 in the second receiving position and it must first be lifted in order to be able to rotate it. This requires a larger force than pure rotation and prevents the culture insert 100 from accidentally falling down into the first receiving position.

The culture insert 100 comprises a bottom 102 that includes a porous membrane. This membrane is permeable to cells and molecules and serves as a scaffold or substrate for cell adhesion, allowing cells to form single- or multi-layered structures on the membrane. These structures should be microscopically examinable. At the same time, nutrient solution can diffuse from the well through the porous membrane and supply the cells with nutrients. The porous membrane has a thickness of between 10 μm and 100 μm, for example 12 μm or 50 μm.

Each of the wells comprises a bottom (see FIG. 6) that is transparent in the visible range and/or in the near-infrared range (in particular up to a wavelength of 1500 nm). Therefore, inverse microscopy can be performed through the bottom. The bottom of the well 12 may comprise or be a cover glass or a plastic, e.g., a polymer film. The optical properties of the bottom correspond in particular to those of a cover glass (e.g. D 263 Schott glass, No. 1.5H). The thickness of the bottom is between 100 μm and 2.0 mm, in particular between 160 μm and 190 μm, further in particular between 165 μm and 175 μm, for example 170 μm. The minimum distance between the cells and a microscope objective is thus the sum of the thickness of the bottom of the well 12 and the thickness of the porous membrane (bottom 102) of the culture insert 100. This circumstance simplifies the use of the device 10 for cultivation and microscopy while simultaneously providing higher optical resolution and, in addition, reduces the risk of possible contamination, as the cells do not have to be removed from the well 12 for microscopy.

In the first receiving position, the small distance between the cells and the objective allows high-resolution microscopy to be performed without having to remove the culture insert 100 from the well 12. In the second receiving position, however, nutrient solution can flow beneath the porous membrane and effectively supply the cells with nutrient solution. Switching between the first receiving position and the second receiving position is particularly easy and user-friendly.

It is therefore not necessary to remove the culture insert 100 from the well in order to switch between the two receiving positions. This also reduces the risk of contamination of the cells and simplifies handling for the user.

FIG. 2 shows a second device 10, which is largely similar to the first device shown in FIG. 1. Identical elements will therefore not be described again. The main difference lies in the design of the positioning element 20.

The positioning element 20 is formed on one edge of well 12 and consists of three pairs of hooks, with the pairs arranged regularly along the edge. In the second receiving position, the culture insert 100 is hooked onto the hooks. In the first receiving position, the culture insert 100 is placed next to the hooks on the edge of the well 12. As in the first device 10 according to FIG. 1, a change from the first to the second receiving position is effected by lifting and turning the culture insert 100. Similarly, the culture insert 100 is locked in the second receiving position because the hooks prevent accidental rotation of the culture insert 100, preventing the culture insert 100 from falling down into the first receiving position.

In general, the positioning element 20 and the culture insert 100 are coordinated with each other in such a way that the two receiving positions can be implemented. The positioning element 20 is specifically adapted to the design of the culture insert 100.

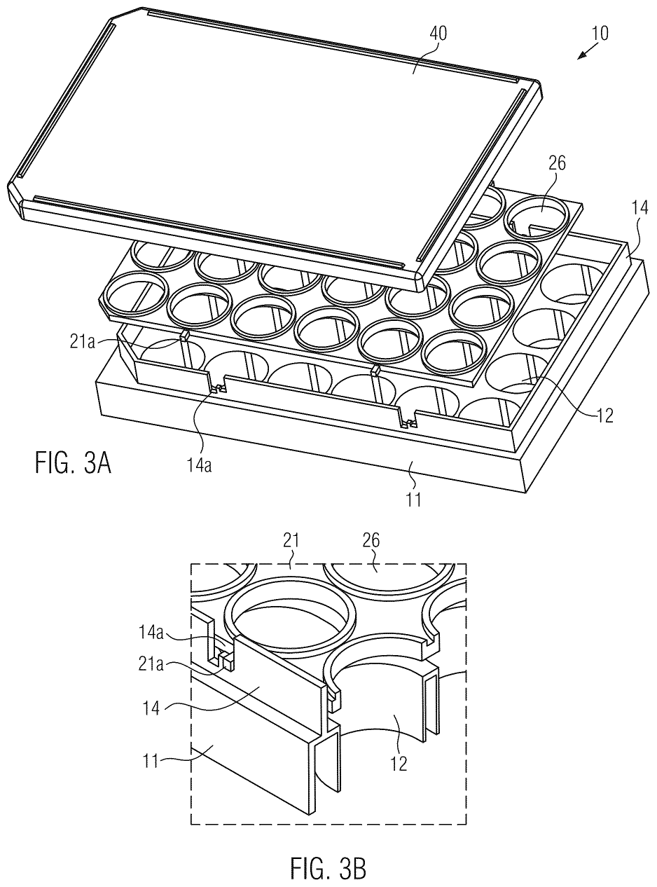

FIG. 3A shows a third form of a device 10, which comprises a different design of the positioning element 21. Here, the positioning element 21 is a plate arranged above the base element 11. The positioning element 21 can be brought into two different positions relative to the base element 11, and these two positions define the first and second receiving positions for the culture insert introduced above. The first position of the positioning element 21 defines the first receiving position of the culture insert 100. The first position of the positioning element 21 defines the first receiving position of the culture insert 100. This is explained in more detail with reference to FIG. 3B. For better illustration of the individual elements, the positioning element 21 is shown in FIG. 3A far above the base element 11 (exploded view).

The positioning element 21 is further formed so that a culture insert can be arranged thereon so that it protrudes into the well 12 in the base element 11. For this purpose, the positioning element has a through hole 26. When the positioning element 21 is arranged in the first position and in the second position above the base element 11, the through hole 26 is located above the well 12 in such a way that the culture insert protrudes into the well 12 through the through hole 26. In particular, the base element 11 is a multi-well plate as described above. Accordingly, the positioning element 21 has as many through holes 26 as the base element 11 has wells. When the positioning element 21 is correctly placed on the base element 11, the through holes 26 are located exactly above the wells 12.

The base element 11 has an upward-facing, circumferential frame that serves as a lateral limiting element 14. This frame restricts the mobility of the positioning element 21 to the extent that only the necessary displacement between the first position and the second position is possible. In other words, the frame prevents the positioning element 21 from being displaced beyond the first and second positions.

FIG. 3B shows a detailed view of how the positioning element 21 is arranged in the second position above the base element 11. The frame has a lateral recess 14a in which two planes are formed. The positioning element 21 has a lateral protrusion 21a which protrudes into the recess 14a and comes to rest on one of the planes. In the second position, the protrusion 21a rests on the higher level. To change to the first position (not shown), the positioning element 21 is displaced and lowered so that the protrusion rests on the lower level. Conversely, to change from the first to the second position, the positioning element 21 is raised and moved.

A raised section may be provided between the two levels of recess 14a to prevent positioning element 21 from accidentally falling down into the first position. In particular, a tight fit can be established in the second position between the positioning element 21 and the base element 11 by clamping the protrusion 21a between the elevation and a side wall of the recess 14a.

One feature of the positioning element 21 in the form of the plate shown is that all culture inserts can be brought simultaneously from the first receiving position to the second receiving position or vice versa. In the devices shown in FIGS. 1 and 2, the culture inserts can be brought individually from the first receiving position to the second receiving position or vice versa. Depending on the desired application, one of the two types of positioning element 20, 21 may be preferred.

The device 10 may also comprise a lid 40 that can be placed on the base element 11. The lid shields the wells 12 from the environment. The lid can be placed on the base element 11 in a form-fitting manner. The positioning element 20 is located in particular between the base element 11 and the lid 40. This allows the samples in the culture insert to be effectively shielded against external environmental influences and contamination. The lid can be placed on all devices described herein in a corresponding manner.

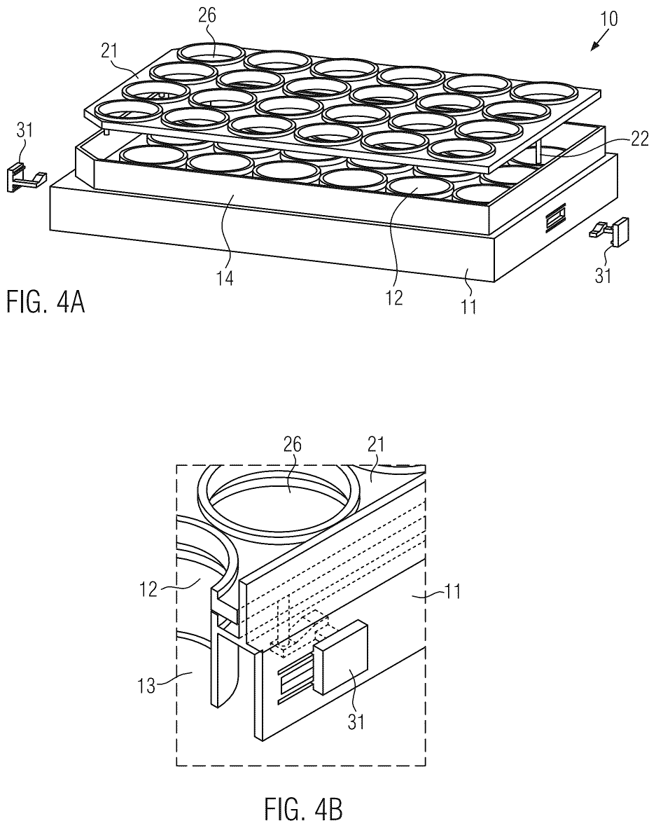

FIG. 4A shows another device 10 with an alternative implementation for switching between the first position and the second position. The base element 11 and positioning element 21 are largely identical to those of the third device, so only the differences are explained here. For better illustration of the individual components, FIG. 4A is an exploded view.

Here, the positioning element 21 has a stand 22 instead of a lateral protrusion (see FIG. 3B). This stand is a column that extends from the surface of the positioning element 21 and stands on the base element 11. The device 10 further comprises a movable ramp as ramp element 31. The ramp element 31 comprises a lower level connected to an upper level via a slope. The positioning element 21 stands on the ramp element 31 via the stand 22. As in FIG. 3A, the device 10 comprises a lateral limiting element 14 in the form of an upward-facing, circumferential frame arranged on the base element 11. Since the change between the two positions of the positioning element 21 is purely vertical here, the frame can fit snugly against the positioning element 21. This prevents unwanted lateral displacement and possible tilting of the positioning element 21.

FIG. 4B is a detailed view of FIG. 4A and shows the positioning element 21 in the first position. The stand 22 is positioned on the lower level of the ramp element 31. When the ramp element 31 is moved, the stand 22 slides up the slope to the upper level, after which the positioning element 21 is in the second position. The movement performed by the positioning element 21 is purely vertical, i.e., without any lateral displacement relative to the base element 11.

To prevent the positioning element 21 from tilting, at least in the second position, two identical ramp elements 31 are provided in the device. These interact with two stands 22 of the positioning element 21, which are arranged on different sides of the latter. Accordingly, the two ramp elements 31 are also arranged on different sides of the base element 11. For optimal switching between the two positions, both ramp elements 31 must be moved synchronously, as otherwise the positioning element 21 may temporarily tilt. The latter can be largely avoided by the tightly fitting frame.

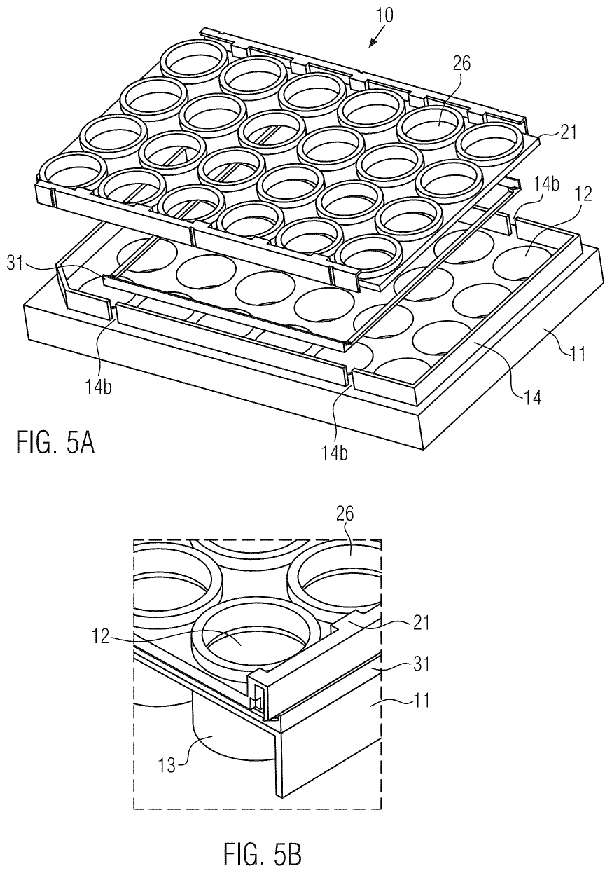

A modification of the fourth device is the fifth device 10 shown in FIGS. 5A and 5B. This differs in particular in the design of the ramp element 31, which will be explained in more detail below. For a better illustration of the individual components, FIG. 5A shows an exploded view.

The ramp element 31 has the shape of a rectangular frame and is referred to below as the positioning frame. The positioning frame is arranged on the base element 11. Similar to FIG. 4A, the base element 11 has a lateral limiting element 14 in the form of a frame, which otherwise has the same properties and functions as explained in FIG. 4A. In addition, the frame comprises four interruptions 14b through which the positioning frame passes. The purpose of this is to ensure that the positioning element can only move along one axis and is otherwise blocked by the frame. The range of this movement is also restricted by the frame. The positioning element 21 in the form of a plate is positioned on the ramp element 31.

The corners of the ramp element 31 feature slopes that enable the positioning element 21 to switch between the first and second positions (see FIG. 4B). For this purpose, one surface of the positioning frame is beveled relative to a horizontal plane in the area of the bevels. A corresponding part of the positioning element rests on this surface. This part can also be beveled at the same angle to achieve a form-fitting connection between the positioning element 21 and the ramp element. Alternatively, this part can also be horizontal.

If the ramp element 31 is now displaced along the aforementioned axis, the positioning element 21 slides over the slope and switches between the first position and the second position. The movement performed by the positioning element 21 is exclusively vertical. The movement performed by the ramp element 31 is exclusively horizontal.

Compared to the fourth device, the fifth device 10 has the advantage that two ramp elements do not have to be moved simultaneously in order to switch between the two positions. However, the ramp element 31 may be more difficult to manufacture in this case. The ramp element 31 may be very delicate, which makes injection molding unreliable. 3D printing offers better results.

Another alternative to the designs shown in FIGS. 4A and 4B is shown in FIG. 6, which is referred to here as the sixth device 10. Here, the ramp (not shown) is a fixed part of the base element 11 and is arranged on a surface thereof. The positioning element 21 comprises a stand (not shown) which, analogous to the case described in FIG. 4B, slides over the ramp when the positioning element 21 is brought from the first position to the second position. The stand may also be designed as in FIG. 4B. However, since the ramp is stationary, the change between the two positions is effected by moving the positioning element 21. The figure now shows the positioning element in the first position (left half of the figure) and the second position (right half) and, associated therewith, a culture insert 100 in the first receiving position (left half) and the second receiving position (right half). As indicated in the figure, a culture insert 100 is arranged on the positioning element 21 so that it protrudes into the well of the base element 11. The culture insert 100 is stationary relative to the positioning element 21.

Belonging to the base element 11, which is a multi-well plate with 24 wells, the positioning element 21 comprises 24 through holes 26. These are located exactly above the wells when the positioning element 21 is placed on the base element. A fastening element 26a is formed at each through hole 26, which is intended for fastening a culture insert with its fastening element 101. This is shown as an example for a through hole 26. There, the culture insert 100 is hooked into the positioning element 21 by engaging the fastening elements 101 of the culture insert 100 in the fastening element 26a at the through hole. The culture insert 100 then protrudes through the through hole 26.

A partition wall 15 is formed between two adjacent wells 12, separating the two wells 12.

Each through hole 26 has a bulge 27, which means that the shape of the through hole 26 deviates from a circular shape. The bulge 27 serves to fill the well with nutrient solution past the culture insert. This can be done with a pipette, for example. The bulge 27 provides additional space for the pipette, making it easier and more convenient to pour in the nutrient solution. This is also shown in the figure.

Furthermore, the device 10 comprises a lateral limiting element 14, which, as shown in FIG. 3A, for example, can be designed as a surrounding frame. The positioning element 21 can only be moved sideways, with the limiting element 14 restricting the sideways movement. When the positioning element 21 hits the right side of the frame, i.e., the right side of the limiting element 14, the first position for the positioning element 21 is reached. The first position defines the first receiving position for the culture insert 100, as also shown in the figure. In the first receiving position, the bottom 102 of the culture insert 100 rests on the bottom 13 of the well 12. As described above, this represents the microscopy position.

When the positioning element 21 strikes the left side of the frame, i.e., the left side of the limiting element 14, the second position for the positioning element 21 is reached. The second position defines the second receiving position for the culture insert 100, as also shown in the figure. In the second receiving position, the bottom 102 of the culture insert 100 is spaced apart from the bottom 13 of the well 12 by a distance which may be, for example, 1 mm. As described above, this represents the cultivation position.

To switch between the first position and the second position, the positioning element 21 must be moved sideways, as can be seen from a comparison of the two relevant partial figures. During this movement, the stand slides over the ramp, thus achieving the vertical offset between the first position and the second position.

When the positioning element 21 strikes the left side of the frame, i.e., the left side of the limiting element 14, the second position for the positioning element 21 is reached. The second position defines the second receiving position for the culture insert 100, as also shown in the figure. In the second receiving position, the bottom 102 of the culture insert 100 is spaced apart from the bottom 13 of the well 12 by a distance which may be, for example, 1 mm. As described above, this represents the cultivation position.

To switch between the first position and the second position, the positioning element 21 must be moved sideways, as can be seen from a comparison of the two relevant partial figures. During this movement, the stand slides over the ramp, thus achieving the vertical offset between the first position and the second position.

This embodiment has several advantages over the devices described above. On the one hand, the change between the two positions 21 can only be made by moving the positioning element 21 sideways, and the vertical offset between the two positions is automatically achieved by moving up or down the ramp. In addition, there is only one point of contact on the positioning element 21 for each direction of change (from the first to the second position and vice versa). This makes the change automatable for a robot, because the robot only needs to be configured to move the positioning element 21 in two opposite directions. It is not necessary to lift or lower the positioning element. Furthermore, as a fixed element of the base element 11, the ramp is easy to manufacture and does not need to be provided as a separate element of the device 10. This is particularly true in comparison to the fifth device shown in FIG. 5A, in which the ramp element has a filigree design and is difficult to manufacture by injection molding.

The base element 11 may be a multi-well plate. In order to fulfill the requirements of the ANSI SLAS standard for multi-well plates (e.g. regarding dimensions and the distance between adjacent wells), the individual wells may have an elliptical cross-sectional area.

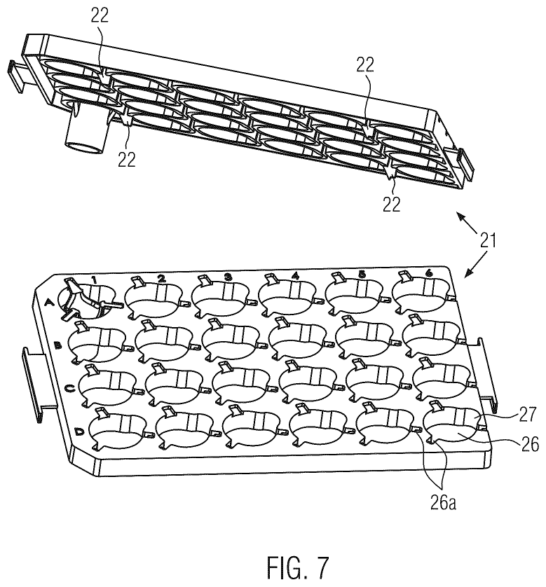

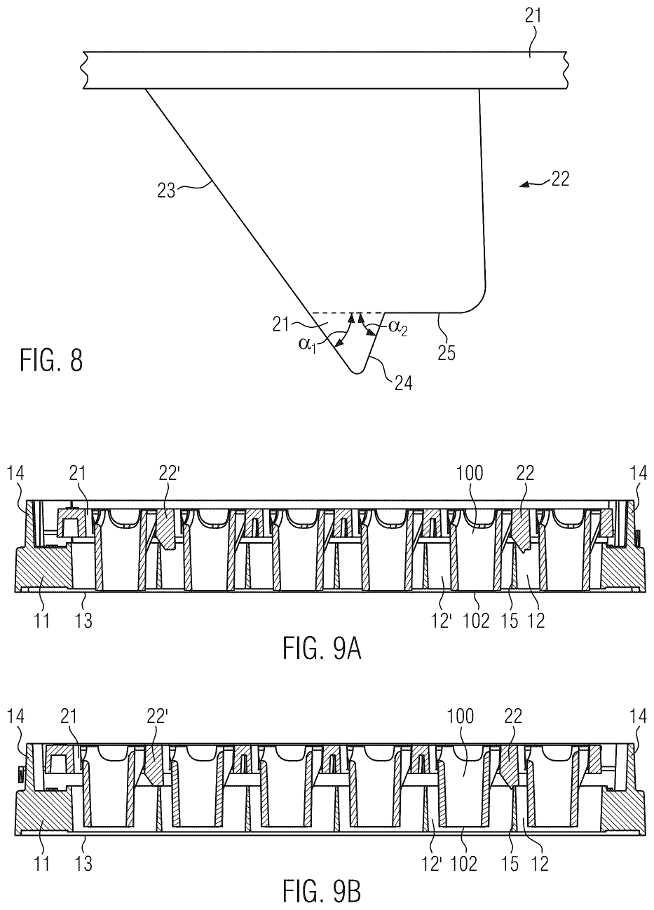

FIG. 7 shows a positioning element 21 that can be combined with a base element 11 according to FIG. 6, but without the ramp, to form a seventh device. The device thus obtained differs from the sixth device according to FIG. 6 in the design of the positioning element 21 and does not include a ramp as part of the base element 11. As in the third to sixth devices described above, the positioning element 21 is a plate that can be arranged on the base element. The through holes 26 and their bulges 27 have also already been described. However, there is a significant difference in the shape of the stand 22, which is now described in more detail with reference to FIG. 8. The positioning element comprises a total of four stands 22. Two are shown directly in the figure, the other two are located in the same configuration on the opposite side of the positioning element 21.

FIG. 8 shows a detailed view of the stand 22 described above, which is arranged on an underside of the positioning element 21. The underside of the stand 22 has three sections: a first section 23, a second section 24, and a third section 25. The first section 23 is beveled or inclined at an angle α1 of 54° to the horizontal. The first section 23 is followed or adjoined by the second section 24, which is beveled or inclined at a second angle α2 of 70° in the opposite direction to the first section. Since the angles are arranged in opposite directions, the two sections 23, 24 form a peak with an angle of 56°. The second section 24 is followed or adjoined by the third section 25, which is essentially formed horizontally. This arrangement results in an elevation, viewed from the third section 25, which is formed by the second 24 and first sections 23. This elevation is also referred to as a tooth. Opposite the third section 25, the tooth has a height of 0.8 mm. The tip of the tooth may be rounded.

FIGS. 9A and 9B show a cross-sectional view of the seventh device 10. As described above, the base element 11 is a multi-well plate with a total of 24 wells 12. This cross-sectional view shows a row of six wells 12. A culture insert 100 is arranged in each well, which in turn is hooked onto the positioning element 21.

FIG. 9A now shows the positioning element 21 in the first position, with the culture inserts 100 accordingly in the first receiving position. In this receiving position, the culture inserts stand on the bottom 13 of the well 12. In other words, the bottom 102 of each culture insert 100 is in contact with the bottom 13 of the respective well 12. The positioning element 21 strikes the lateral limiting element 14 on the right-hand side, so that the positioning element 21 cannot be moved further to the right. In this first position, the first section 23 of the stand 22 is in contact with an edge of the well 12. Since the positioning element 21 strikes the lateral limiting element 14, it cannot slide any further, even though the beveled first section 23 is resting on the edge.

The positioning element 21 can be lowered further in the first position than defined by the first receiving position. The difference between the corresponding first position and the actual first position is referred to as stroke oversize. This stroke oversize ensures that all culture inserts 100 in a multi-well plate are in contact with the bottom 13 of the well 12. Any deviations in the culture inserts 100 or the positioning element 21 can thus be compensated for. The stroke oversize can be between 0.3 mm and 0.5 mm, for example 0.4 mm.

FIG. 9B now shows the positioning element 21 in the second position, with the culture inserts 100 accordingly in the second receiving position. Here, there is a distance between the bottom 13 of the well 12 and the bottom 102 of the culture insert 100. This distance can be, for example, 1 mm or more. The positioning element 21 strikes the lateral limiting element 14 on the left side, so that the positioning element 21 cannot be moved further to the left. In the second position of the positioning element 21, the third section 25 rests on the edge of the well 12. More precisely, there is a partition wall 15 between the two wells 12, 12′, which separates the two wells 12, 12′, and the third section 25 rests on this partition wall 15. The tooth protrudes into the adjacent well 12′.

To move the positioning element into the first position, it may be shifted to the right to achieve the configuration shown in FIG. 9A. To do this, the tooth may be moved from the adjacent well 12′ into the well 12 by sliding the second section 24 over the edge of the adjacent well 12′ by moving the positioning element 21, thus overcoming the tooth, after which the first section 23 is in contact with the edge of the well 12. The tooth and the second section 24 therefore may serve the purpose of ensuring that the positioning element 21 does not simply move from the second to the first position, but that additional force may be required.

The angles of the sections shown in FIG. 8 may be optimal. The first angle α1 is small enough to allow the positioning element 21 to be moved with reasonable force, and large enough to overcome the tooth within the available space for lateral movement, thus achieving the lateral offset between the first position and the second position. The second angle α2 is small enough to allow the positioning element 21 to be moved back to the first position by a robotic drive, and large enough to prevent the positioning element 21 from being accidentally moved to the first position.

The ramp concept described above for easy and automated switching between the two positions of the positioning element is also included in this embodiment. The first section 23 serves as a ramp over which the positioning element 21 may be pushed upwards in order to switch between the first position and the second position. The main purpose of the second section 24 may be to lock the positioning element 21 in the second position so that it cannot accidentally slide down into the first position. Nevertheless, the change between the two positions can be accomplished by lateral displacement of the positioning element 21. This has the advantage that the change can be automated relatively easily. In particular, a robot configured accordingly can perform the change, and the robot may not have to perform any complex movements to make the change. This also allows long-term cultures to be cultivated and observed automatically. In such long-term cultures, microscopic observations may be made in the meantime, which may require a change from the second to the first position. After microscopy, the positioning element may be returned to the second position for microscopy. This can now be completely automated, which facilitates the practicability of corresponding experiments and test series.

In the cross-sectional view shown, one of the stands 22 has the special structure with three sections, which was explained in more detail with reference to FIG. 8. A second stand 22′ comprises only two sections, which are equivalent to the first and third sections. In total, the positioning element 21 comprises four stands, two of which have the structure with the three sections 23, 24, 25. This is sufficient to achieve the advantages described. Since the three sections are delicate and difficult to manufacture, it may be advantageous to design only two of the stands 22 accordingly.

1. A device for cultivating cell biological samples in a culture insert, the device comprising:
a base element in which a well with a bottom is formed, wherein the well is configured for arranging the culture insert in the well; and
a positioning element configured to provide a first receiving position and a second receiving position for the culture insert,
wherein the culture insert is arranged in one of the first receiving position or in the second receiving position on the positioning element so that the culture insert is arranged in the well, and
wherein, in the first receiving position, a distance between the bottom of the well and a bottom of the culture insert is smaller than in the second receiving position.

2. The device according to claim 1, wherein the positioning element has two different levels that define the first receiving position and the second receiving position, and
wherein the positioning element is formed such that a change between the first receiving position and the second receiving position is effected by a relative movement between the positioning element and the culture insert.

3. The device according to claim 1, further comprising a base plate, wherein the positioning element is movable relative to the base plate and is configured to be brought into a first position and a second position, wherein the first position defines the first receiving position and wherein the second position defines the second receiving position for the culture insert.

4. The device according to claim 3, further comprising a ramp
wherein the positioning element is configured to be brought from the first position to the second position via the ramp.

5. The device according to claim 4, wherein the ramp is an additional movable element of the device configured to be displaced relative to the base element, and
wherein the positioning element is configured to be brought from the first position to the second position by displacing the ramp.

6. The device according to claim 4, wherein the positioning element is arranged above the base element,
wherein the ramp is arranged between the base element and the positioning element, and
wherein the positioning element is in contact with the ramp.

7. The device according to claim 3, wherein the positioning element is arranged above the base element and comprises a stand with an at least partially beveled underside,
wherein a beveled portion of the underside of the stand is in contact with an edge of the well and partially protrudes into the well when the positioning element is in the first position.

8. The device according to claim 1, wherein the culture insert is configured to be locked in the second receiving position by an increased force to move the culture insert into the first receiving position.

9. The device according to claim 7, wherein the underside of the stand comprises:
a first section that is inclined at a first angle relative to a horizontal plane;
a second section that adjoins the first section and is inclined at a second angle, which is opposite in sign to the first angle, to the horizontal plane; and
a third section that adjoins the second section and is formed parallel to the horizontal plane,
wherein in the first receiving position, the first section is in contact with the edge of the well, and
wherein, in the second receiving position, the third section is in contact with the edge of the well.

10. The device according to claim 9, wherein at least one of:
the first angle is between 50° and 60°, or
the second angle is between 65° and 75°.

11. The device according to claim 3, further comprising at least one lateral limiting element for limiting a horizontal movement of the positioning element between the first position and the second position.

12. The device according to claim 3, wherein the positioning element has a through hole which is formed for arranging of the culture insert.

13. The device according to claim 9, wherein the positioning element has two stands with at least one of the first section, the second section, or the third section.

14. A system for cultivating biological samples, comprising:
a culture insert; and
a device comprising:
a base element in which a well with a bottom is formed, wherein the culture insert is to be arranged in the well; and
a positioning element configured to provide a first receiving position and a second receiving position for the culture insert,
wherein the culture insert is arranged in one of the first receiving position or in the second receiving position on the positioning element so that the culture insert is arranged in the well, and
wherein, in the first receiving position, a distance between the bottom of the well and a bottom of the culture insert is smaller than in the second receiving position.

15. The system according to claim 14, wherein the culture insert comprises:
a base with a porous membrane that is permeable to cells or molecules; and
a fastening element configured to arrange the culture insert on the positioning element.

16. The device according to claim 1, wherein the positioning element is configured to be locked in the second position by an increased force to move the positioning element into the first position.

17. The device according to claim 10, wherein the first angle is approximately 54° and wherein the second angle is approximately 70°.

18. The device according to claim 12, wherein the through hole comprises a bulge, and wherein a shape of the through hole deviates from a circular or elliptical shape due to the bulge.

19. The system according to claim 14, wherein the positioning element has two different levels that define the first receiving position and the second receiving position, and
wherein the positioning element is formed such that a change between the first receiving position and the second receiving position is effected by a relative movement between the positioning element and the culture insert.

20. The system according to claim 14, wherein the device further comprises a base plate, wherein the positioning element is movable relative to the base plate and is configured to be brought into a first position and a second position, wherein the first position defines the first receiving position and wherein the second position defines the second receiving position for the culture insert.
